(19) United States
(12) Patent Application Publication
(10) Pub. No.: US 2024/0307714 A1
(43) Pub. Date: Sep. 19, 2024

(54) Personal Air Purifier

(71) Applicant: John Raymond Yick Won Chan, FREMONT, CA (US)

(72) Inventor: John Raymond Yick Won Chan, FREMONT, CA (US)

(21) Appl. No.: 18/605,410

(22) Filed: Mar. 14, 2024

Related U.S. Application Data
(60) Provisional application No. 63/490,026, filed on Mar. 14, 2023.

(51) Int. Cl. A62B 23/02; A62B 18/00; B03C 3/017; B03C 3/019; B03C 3/04; B03C 3/36; B03C 3/41; B03C 3/82

(52) U.S. Cl. CPC A62B 23/02; A62B 18/003; B03C 3/017; B03C 3/019; B03C 3/04; B03C 3/368; B03C 3/41; B03C 3/82

(57) ABSTRACT

A personal air purifier is a portable device that eliminates all pathogens and particulates present in the air breathed by the user. The device includes a purifier housing, an air inlet, an air outlet, a metallic air path, an air ionizer, a power source, an air fan, and a controller. The purifier housing retains the metallic air path, the air ionizer, the power source, the air fan, and the controller. The air inlet and the air outlet are openings that allow airflow through the purifier housing. The air fan generates airflow through the purifier housing. The metallic air path and the air ionizer sanitize the airflow through the purifier housing. The power source provide the operating power for the air ionizer, the air fan, and the controller. The power source positively charges the metallic air path. The controller enables the selective control of the air ionizer and the air fan.

### FIELD OF THE INVENTION

The present invention relates generally to air filtration systems and Personal Protective Equipment (PPE). More specifically, the present invention discloses a portable air purifier designed for personal use.

### BACKGROUND OF THE INVENTION

There are over thirty types of diseases caused by various airborne bacteria and viruses. These include influenza, Covid-19, respiratory syncytial virus pneumonia, acute flaccid myelitis, anthrax, chickenpox, meningitis, measles, Middle East respiratory syndrome, multisystem inflammatory syndrome, mumps, pertussis, plaque, pneumococcal diseases, severe acute respiratory syndrome, and tuberculosis. These airborne diseases have caused the suffering of billions of people, millions of deaths, and an enormous impact on the economy. However, it is almost impossible to come up with vaccines and therapeutics to treat all these diseases that can be distributed to the world population of over eight billion people of different ages and health conditions without any side effects. For example, the Covid-19 corona virus mutates every few months, thus creating a moving target. Therefore, the best solution is to eliminate all airborne bacteria and viruses before people are exposed to them.

An objective of the present invention is to provide a personal air purifier that eliminates all airborne pathogens including particulate matter present in the air before entering the human body. The present invention is a wearable device that can be carried along by the user to always protect the user from bacteria and viruses. Further, another objective of the present invention is to provide a personal air purifier that provides greater protection to the user from airborne pathogens than other similar Personal Protective Equipment (PPE), such as face masks. Additional features and benefits of the present invention are further discussed in the sections below.

### SUMMARY OF THE INVENTION

The present invention discloses a personal air purifier. The personal air purifier is a wearable device that protects the user from airborne pathogens and particulates by providing purified air that the user can always breathe. To do so, the personal air purifier preferably includes purifier housing at least one air inlet and at least one air outlet through which airflow enters and exits the purifier housing, respectively. The present invention may also include a fan pump that drives the airflow through the purifier housing. Further, the present invention can include various air sanitation features that remove the pathogens and other particulates from the airflow through the purifier housing. For example, the present invention can include a metallic air path that enables an oligodynamic effect to attract and trap pathogens present in the airflow through the purifier housing. The metallic air path can be negatively charged to facilitate the attraction of pathogens.

Further, the metallic surfaces of the metallic air path can be texturized to facilitate the trapping of the pathogens. The trapped pathogens then naturally die over time without possibility of being released back into the airflow through the purifier housing. In addition, the present invention can include an air ionizer that ionizes the airflow through the purifier housing to further purify the airflow through the purifier housing. As a result, purified air is generated which exits through the at least one air outlet and is guided towards the user. In some embodiments, the present invention can be designed to output the purified air directly towards the user's face. In other embodiments, the present invention can be provided with a face mask that guides the airflow of the generated purified air towards the face of the user. The face mask can be designed as a full-face mask that fully covers the user's face or a nose-mouth mask that only covers the user's mouth and nose.

### BRIEF DESCRIPTION OF THE DRAWINGS

FIG. 1 is a top-front perspective view of the present invention, wherein the tubing is shown connecting the purifier housing to the face mask, and wherein the tubing is shown with a symbolic break in its length.

FIG. 2 is a side view of the present invention, wherein the tubing is shown with a symbolic break in its length.

FIG. 3 is a top-front perspective view of the purifier housing of the present invention.

FIG. 4 is a bottom-rear perspective view of the purifier housing of the present invention.

FIG. 5 is a top-front-exploded perspective view of the purifier housing of the present invention.

FIG. 6 is a bottom-rear-exploded perspective view of the purifier housing of the present invention.

FIG. 7 is a top-front perspective view of a first embodiment of the purifier housing of the present invention.

FIG. 8 is a bottom-rear perspective view of the first embodiment of the purifier housing of the present invention.

FIG. 9 is a top-front-exploded perspective view of the first embodiment of the purifier housing of the present invention.

FIG. 10 is a bottom-rear-exploded perspective view of the first embodiment of the purifier housing of the present invention.

FIG. 11 is a top-front-exploded perspective view of a second embodiment of the purifier housing of the present invention.

FIG. 12 is a bottom-rear-exploded perspective view of the second embodiment of the purifier housing of the present invention.

FIG. 13 is a front view of the metallic air path of the present invention.

FIG. 14 is a magnified view of the plurality of metal sheets of the metallic air path of the present invention, wherein the metallic surfaces of the plurality of metal sheets are shown with different textures.

FIG. 15 is a magnified view of the metallic surfaces of the plurality of metal sheets of the present invention, wherein the metallic surfaces are shown covered with a quantity of conductive lubricant.

FIG. 16 is a top-front perspective view of the face mask of the present invention, wherein the tubing is shown connecting the purifier housing to the air nozzle, and wherein the tubing is shown with a symbolic break in its length.

FIG. 17 is a top-front perspective view of the face mask of the present invention.

FIG. 18 is a top-front perspective view of an alternate embodiment of the face mask of the present invention.

FIG. 19 is a schematic view showing the electrical connections and the electronic connections of the present invention, wherein the electrical connections are shown in solid line, and wherein the electronic connections are shown in dotted lines.

### DETAIL DESCRIPTIONS OF THE INVENTION

All illustrations of the drawings are for the purpose of describing selected versions of the present invention and are not intended to limit the scope of the present invention.

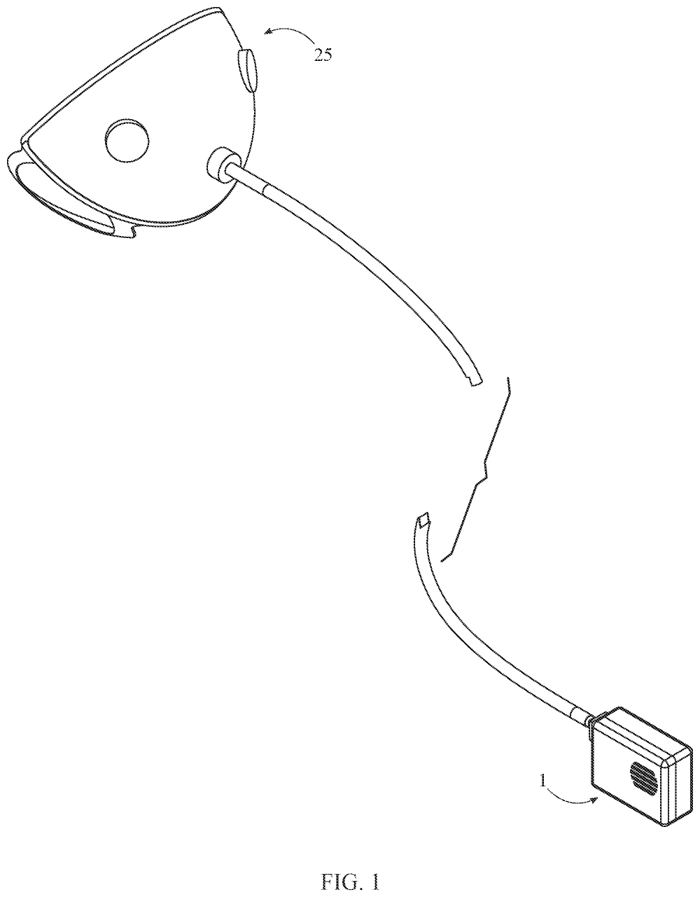

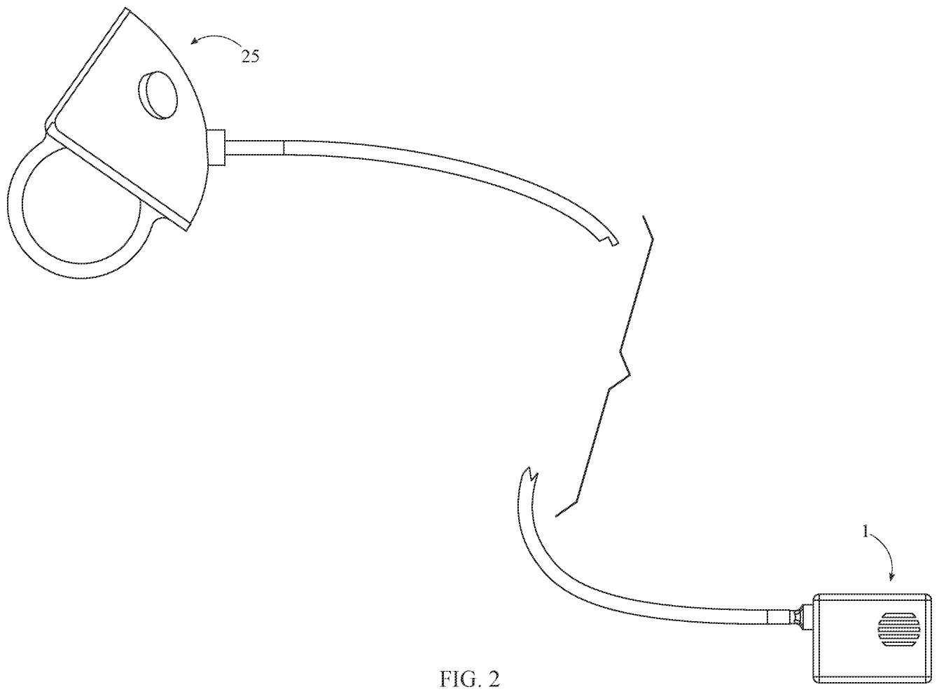

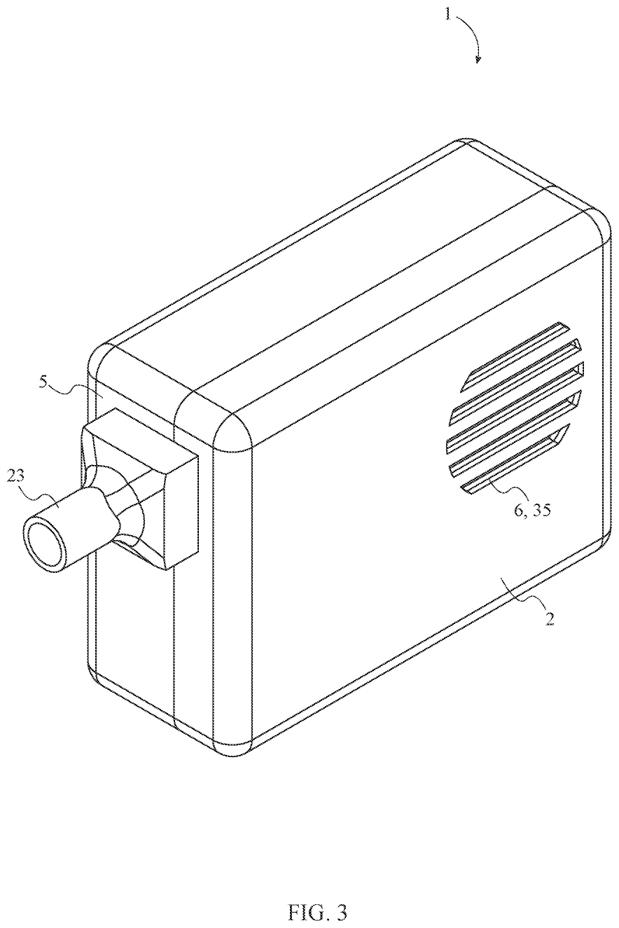

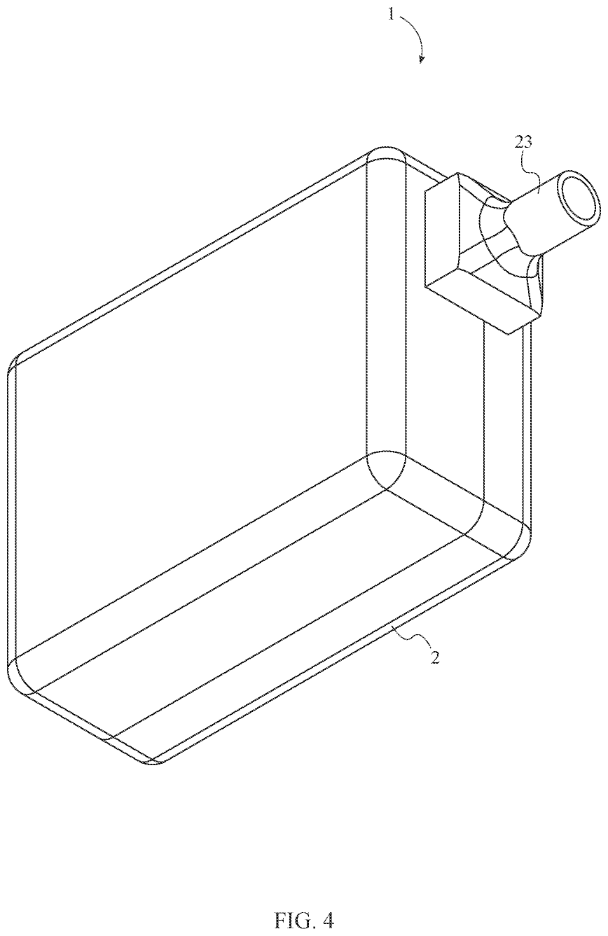

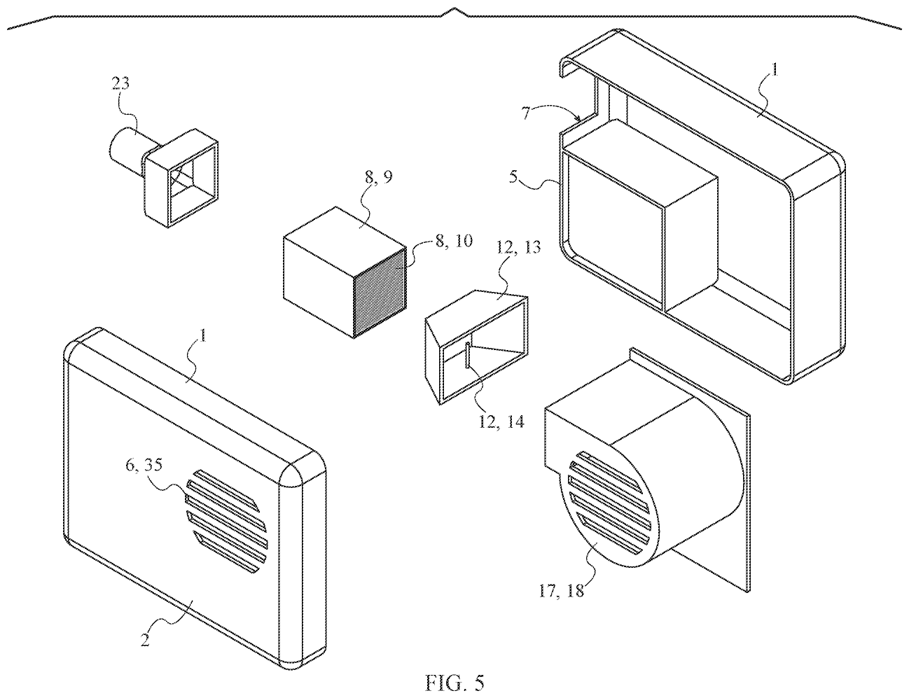

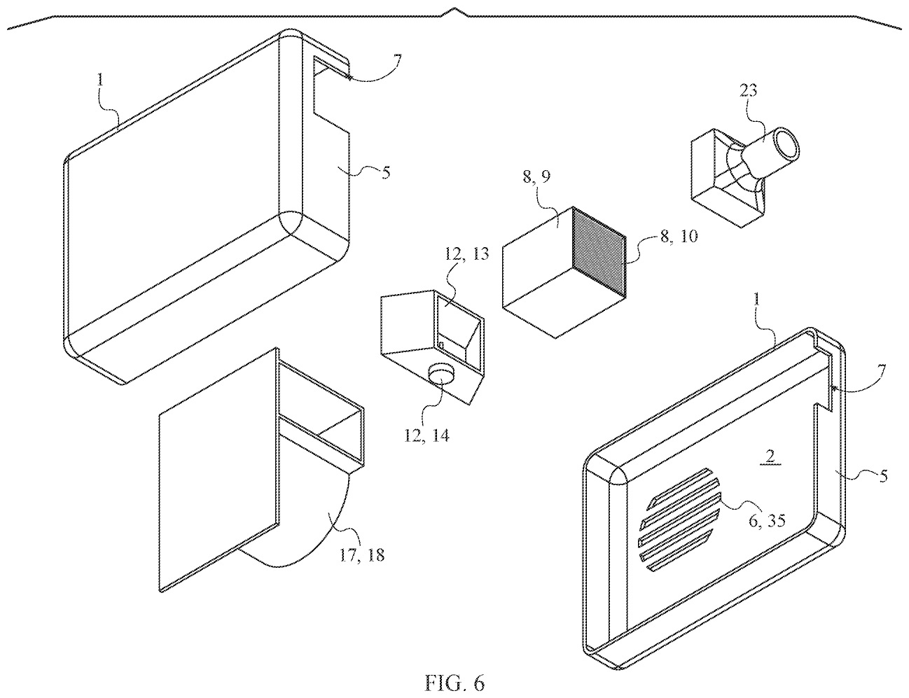

The present invention discloses a personal air purifier. The personal air purifier is a portable device that eliminates all pathogens and particulates present in the air being breathed by the user. As can be seen in FIG. 1 through 6, the present invention comprises a purifier housing 1, at least one air inlet 6, at least one air outlet 7, a metallic air path 8, an air ionizer 12, a power source 15, an air fan 17, and a controller 19. The purifier housing 1 corresponds to the structure that retains the metallic air path 8, the air ionizer 12, the power source 15, the air fan 17, and the controller 19. The at least one air inlet 6 and the at least one air outlet 7 correspond to openings on the purifier housing 1 that allow airflow through the purifier housing 1. The air fan 17 generates airflow through the purifier housing 1. The metallic air path 8 and the air ionizer 12 both sanitize the airflow through the purifier housing 1 to generate purified airflow. The power source 15 provide the power necessary for the air ionizer 12, the air fan 17, and the controller 19 to operate. The power source 15 also provides the charge necessary to positively charge the metallic air path 8. Furthermore, the controller 19 enables the user to selectively control the operation of the air ionizer 12 and the air fan 17.

The general configuration of the aforementioned components enables the user to breathe purified anywhere at any time. As can be seen in FIG. 1 through 6, the purifier housing 1 is preferably a portable structure large enough to be comfortably carried by the user. The purifier housing 1 is preferably a hollow rectangular-shaped structure that can be carried on the user without obstructing the user's body movement. However, the purifier housing 1 can be shaped differently to accommodate different design parameters. The at least one air inlet 6 and the at least one air outlet 7 are externally integrated onto the purifier housing 1 to allow airflow through the purifier housing 1. For example, the at least one air inlet 6 can be an opening on the side of the purifier housing 1 large enough to allow the desired amount of air to flow into the purifier housing 1. The at least one air outlet 7 can be an adjustable air vent that outputs purified air in the desired direction. Further, the metallic air path 8, the air ionizer 12, the power source 15, the air fan 17, and the controller 19 are mounted within the purifier housing 1 to protect these components from the surroundings. The purifier housing 1 can be designed as a modular structure that can be opened to allow for maintenance or repair. For example, the purifier housing 1 can include a removable panel that can be opened to access the interior of the purifier housing 1, such as a side panel. In addition, the at least one air inlet 6 is in fluid communication with the at least one air outlet 7 through the air fan 17, through the air ionizer 12, and through the metallic air path 8, so that the airflow through the purifier housing 1 is sanitized by the air ionizer 12 and the metallic air path 8.

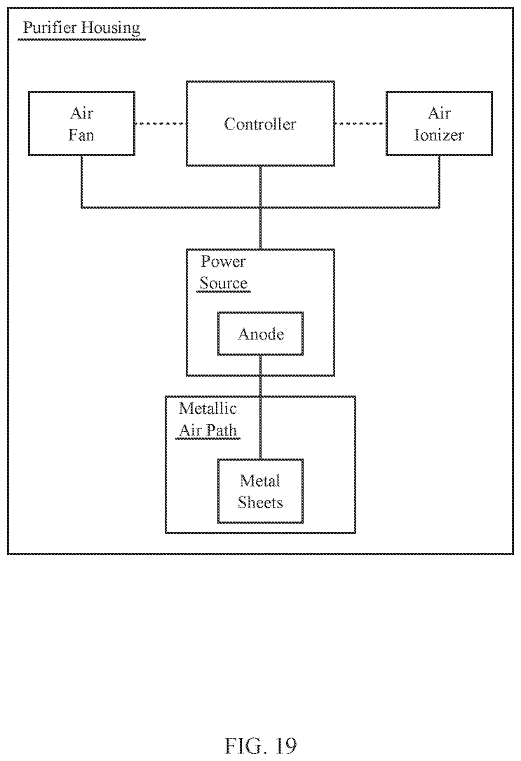

As can be seen in FIGS. 1 through 6 and 19, the air ionizer 12 is designed to negatively charge pathogen and other particulates so that the charged particulates are attracted to the metallic air path 8 where the charged particulates are trapped. In addition, the air ionizer 12 and the metallic air path 8 can be arranged into different configurations to sanitize the airflow through the purifier housing 1. Further, the controller 19 is electronically connected to the air ionizer 12 and the air fan 17 so that the user can selectively activate the air fan 17 and the air ionizer 12 to start generating a purified airflow. For example, the controller 19 may include a power button externally integrated into the purifier housing 1 to allow the user to selectively power the device on/off. In addition, the power source 15 is electrically connected to the controller 19, the air ionizer 12, and the air fan 17 to provide the power necessary for the operation of the controller 19, the air ionizer 12, and the air fan 17. Further, the metallic air path 8 is electrically connected to an anode 16 of the power source 15 so that the metallic air path 8 is positively charged, thus attracting the ionized charged pathogens and particulates from the air ionizer 12.

In some embodiments, the at least one air inlet 6 can also be covered by a covering with several openings to allow airflow through the at least one air inlet 6 while also blocking larger objects from entering the purifier housing 1 which can damage or hinder the operation of the present invention. As can be seen in FIG. 1 through 6, the present invention may further comprise an air vent 35 that covers the at least one air inlet 6 while enabling airflow into the purifier housing 1. The air vent 35 may include any appropriate design that does not restrict the airflow into the purifier housing 1. The air vent 35 is integrated into the at least one air inlet 6 so that the air vent 35 is secured to the at least one air inlet 6. Similarly, the at least one air outlet 7 can be an opening on the side of the purifier housing 1 that allows different attachments to be connected to the purifier housing 1.

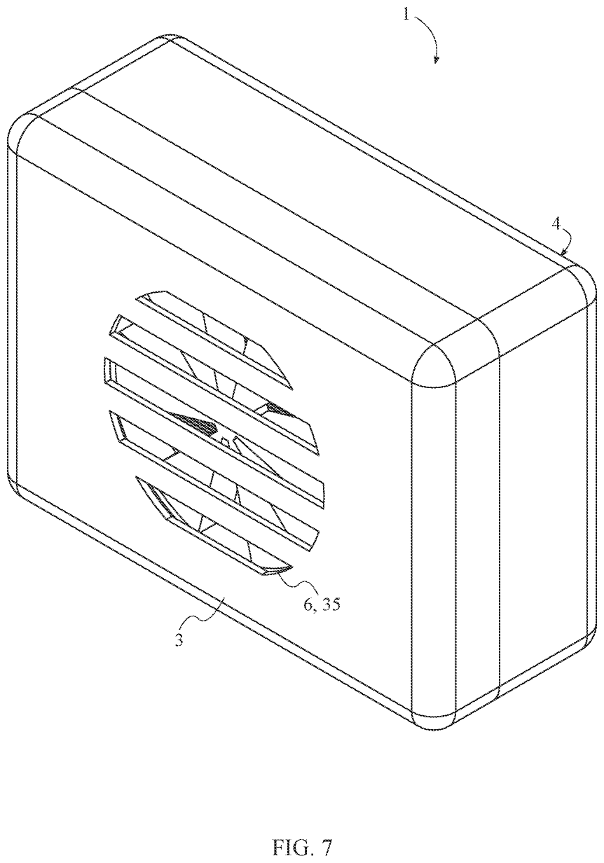

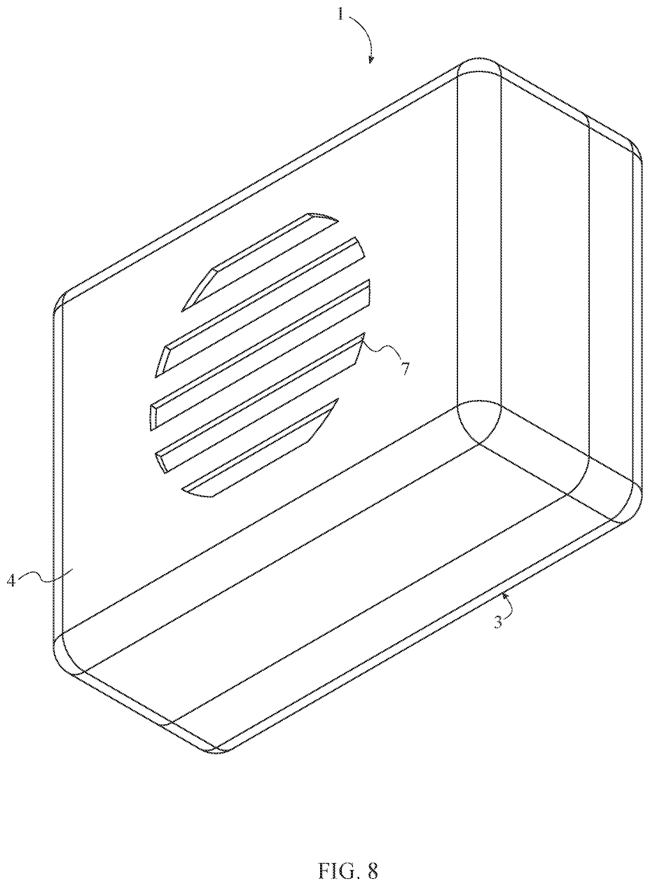

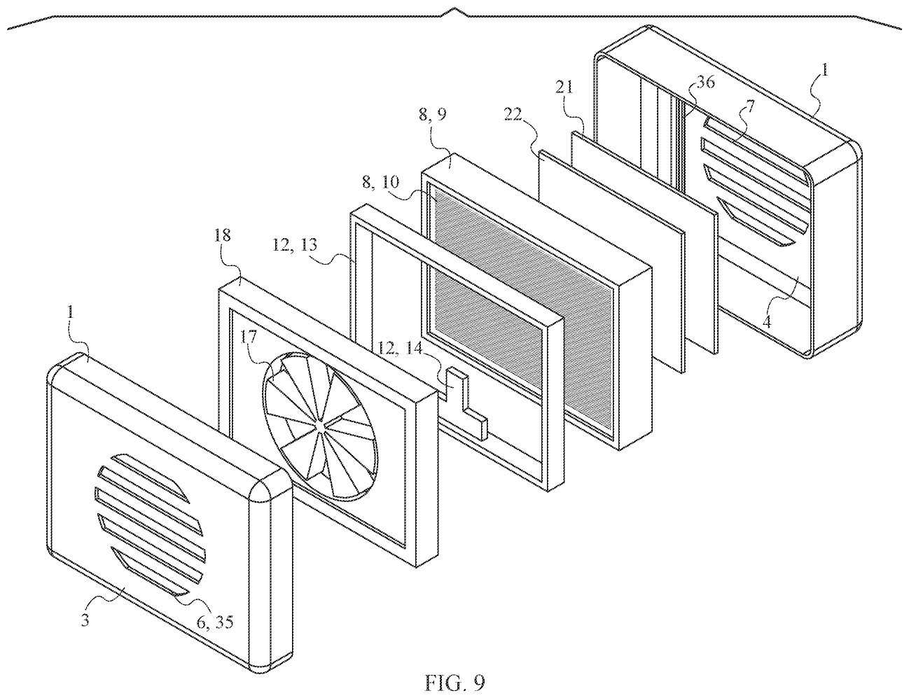

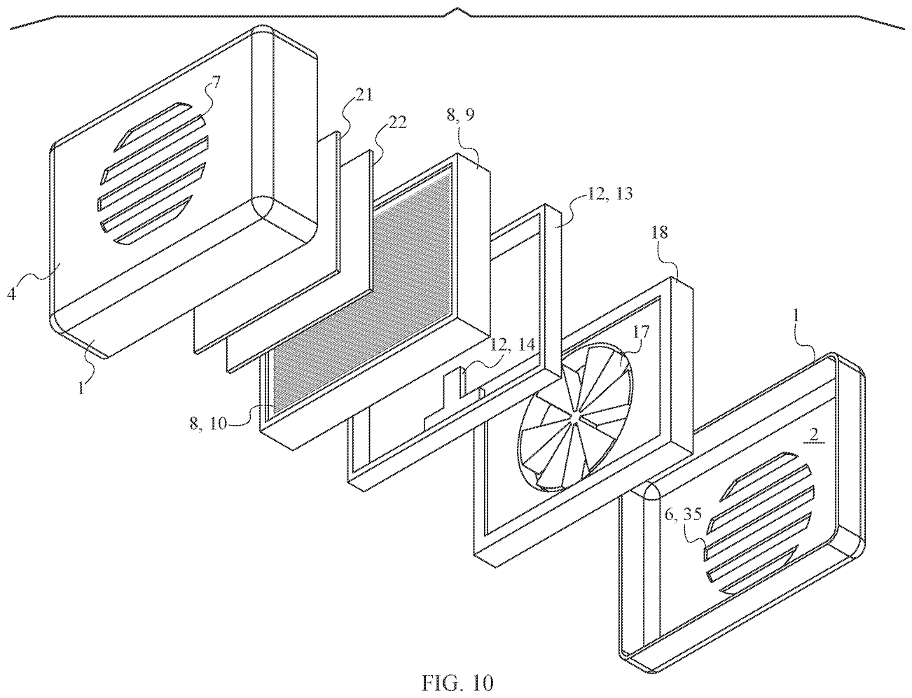

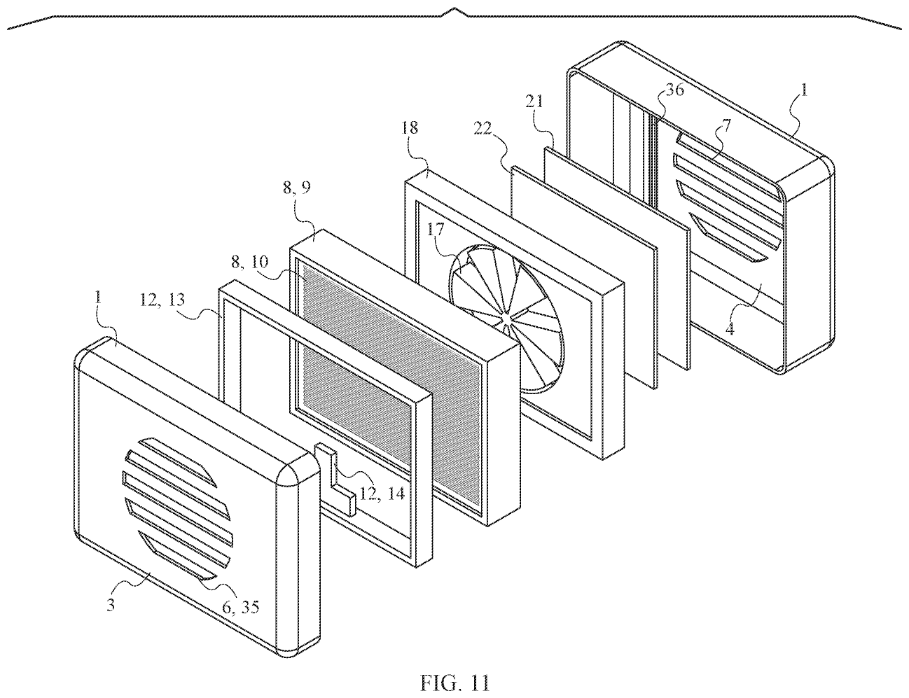

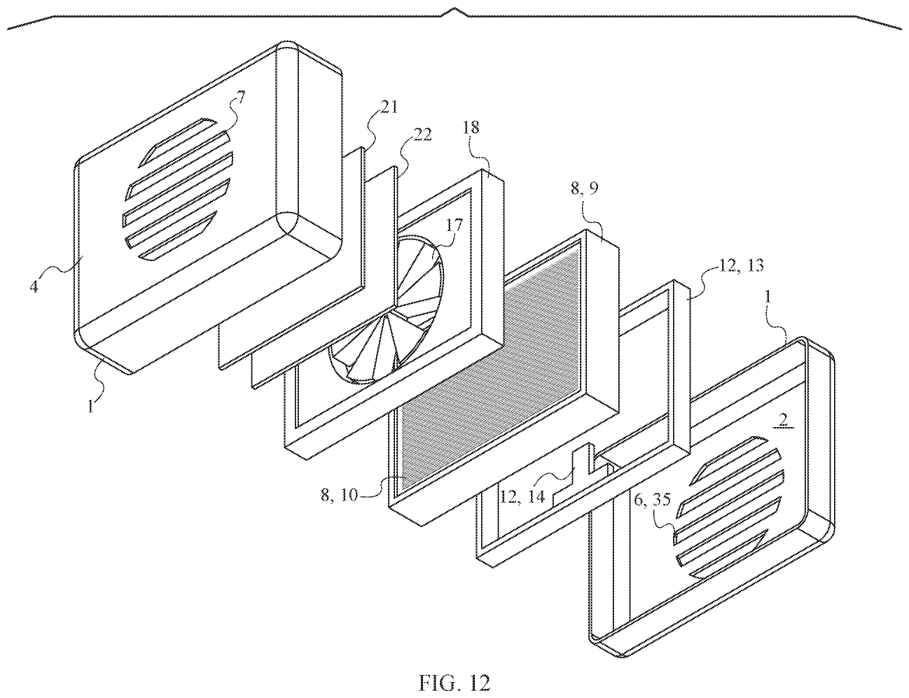

The at least one air inlet 6 and the at least one air outlet 7 can be arranged in different configurations according to the design of the purifier housing 1. As can be seen in FIG. 7 through 10, in one embodiment, the purifier housing 1 may comprise at least one lateral panel 2 and at least one perimeter panel 5. The at least one lateral panel 2 can be any lateral panel of the rectangular-shaped purifier housing 1, while the at least one perimeter panel 5 can be any perimeter panel. Further, the at least one air inlet 6 is integrated into the at least one lateral panel 2, while the at least one air outlet 7 is integrated into the at least one perimeter panel 5. This way, an indirect airflow is generated through the purifier housing 1. As can be seen in FIGS. 11 and 12, in another embodiment, the purifier housing 1 may comprise a first lateral panel 3 and a second lateral panel 4. The first lateral panel 3 and the second lateral panel 4 can be lateral panels of the rectangular-shaped purifier housing 1 positioned opposite to each other. Further, the at least one air inlet 6 is integrated into the first lateral panel 3, while the at least one air outlet 7 is integrated into the second lateral panel 4. This way, a direct airflow is generated through the purifier housing 1. However, depending on the different designs of the purifier housing 1, the at least one air inlet 6 and the at least one air outlet 7 can be positioned in different locations around the purifier housing 1.

As previously discussed, the air fan 17, the air ionizer 12, and the metallic air path 8 can be arranged into different configurations according to the purifier housing 1. As can be seen in FIG. 1 through 6, regardless of the arrangement, the air fan 17 may comprise a fan housing 18 that facilitates the generation of the airflow by the air fan 17. As can be seen in FIG. 7 through 10, in one embodiment, the air fan 17 can be arranged to directly draw surrounding air into the purifier housing 1 to generate an airflow through the purifier housing 1. The fan housing 18 is in fluid communication with the at least one air inlet 6 to directly draw surrounding air into the purifier housing 1. In addition, the metallic air path 8 is in fluid communication with the at least one air outlet 7 so that the airflow through the purifier housing 1 passes through the metallic air path 8 before exiting the purifier housing 1. Further, the fan housing 18 is in fluid communication with the metallic air path 8 through the air ionizer 12 so that the air drawn into the purifier housing 1 by the air fan 17 passes through the air ionizer 12 first before passing through the metallic air path 8.

As can be seen in FIGS. 11 and 12, in another embodiment, the air fan 17 can be arranged to indirectly draw surrounding air into the purifier housing 1 to generate an airflow through the purifier housing 1. The air ionizer 12 is in fluid communication with the at least one air inlet 6 so that surrounding air drawn into the purifier housing 1 first passes through the air ionizer 12. The fan housing 18 is in fluid communication with the at least one air outlet 7 so that the air fan 17 draws the airflow through the purifier housing 1 by directly moving the airflow out through the at least one air outlet 7. Further, the air ionizer 12 is in fluid communication with the fan housing 18 through the metallic air path 8 so that once the drawn air passes through the air ionizer 12, the ionized airflow passes through the metallic air path 8. The sanitized air is then moved out of the purifier housing 1 through the at least one air outlet 7. In other embodiments, different arrangements of the air ionizer 12, the metallic air path 8, the air fan 17 can be implemented within the purifier housing 1 differently to accommodate different designs of the purifier housing 1.

In some embodiments, to ensure that the generated airflow through the purifier housing 1 is ionized, the air ionizer 12 may comprise an ionizer chamber 13 and at least one electrode 14, as can be seen in FIG. 1 through 12. The at least one electrode 14 emits electrons within the ionizer chamber 13 to ionize the airflow. The ionizer chamber 13 ensures that the airflow is exposed to the at least one electrode 14 during the ionization process. Further, the at least one electrode 14 is mounted within the ionizer chamber 13 to secure the at least one electrode 14 within the ionizer chamber 13. In addition, the ionizer chamber 13 is in fluid communication with the metallic air path 8 so that the airflow being ionized is guided into the metallic air path 8. In other embodiments, different configurations can be implemented for the air ionizer 12.

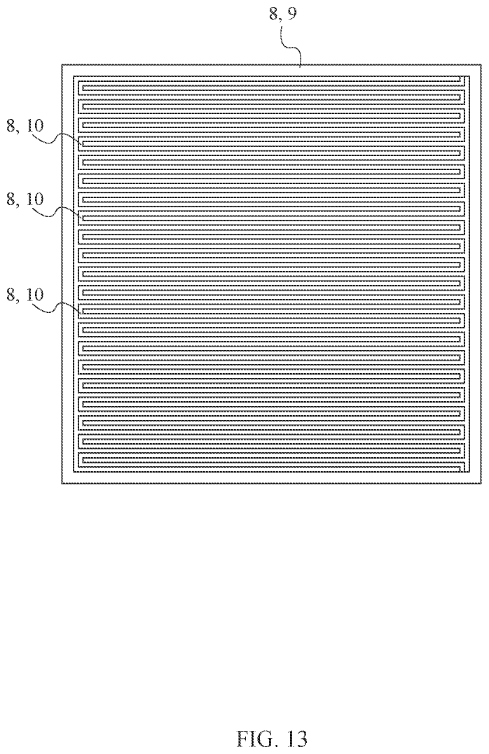

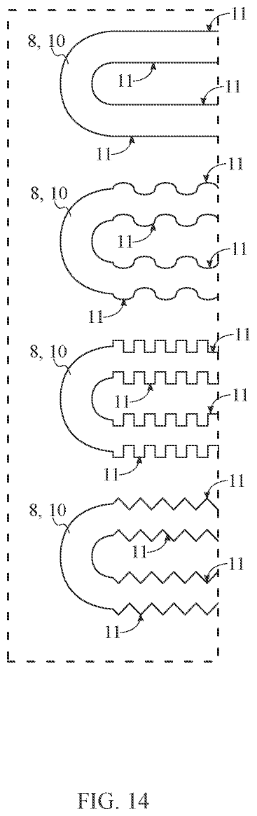

As previously discussed, the metallic air path 8 is designed to attract the ionized particulates in the airflow that has passed through the air ionizer 12. As can be seen in FIG. 13 through 15, the metallic air path 8 is designed to increase the exposure of the ionized airflow to the metallic surfaces 11 of the metallic air path 8 so that all the ionized particulates are attracted and trapped by the metallic surfaces 11 of the metallic air path 8. So, the metallic air path 8 may comprise a tubular housing 9 and a plurality of metal sheets 10. The tubular housing 9 is preferably an open-ended tubular structure that retain the plurality of metal sheets 10. The plurality of metal sheets 10 corresponds to several metal sheets shaped into a multi-compartment structure through which the ionized airflow passes. Further, the plurality of metal sheets 10 is mounted within the tubular housing 9 to secure the plurality of metal sheets 10 is secured to the tubular housing 9. In addition, each of the plurality of metal sheets 10 is positioned parallel to each other so that the plurality of metal sheets 10 do not intersect each other. Each of the plurality of metal sheets 10 is also positioned offset to each other to provide enough space for the airflow to pass through the metallic air path 8. In some embodiments, the plurality of metal sheets 10 can be a continuous sheet of metal bent into a short-stacked winding/serpentine path resembling a radiator heat exchanger. However, other shapes or patterns can be implemented for each of the plurality of metal sheets 10 to increase the exposure of the ionized airflow to the metallic surfaces 11 of the plurality of metal sheets 10 within the tubular housing 9.

As can be seen in FIG. 13 through 15, further, the plurality of metal sheets 10 can be specially made to help eliminate the trapped ionized particulates. Each of the plurality of metal sheets 10 is preferably made of an oligodynamic metal that help the plurality of metal sheets 10 to have a biocidal effect to eliminate the trapped ionized particulates. For example, the plurality of metal sheets 10 can be made of mercury, silver, copper, bronze, brass, lead, iron, bismuth, or a combination thereof. On the other hand, the tubular housing 9 is preferably made of a plastic material that acts as an insulator to prevent the positive charge on the plurality of metal sheets 10 from travelling to other metallic components within the purifier housing 1. Furthermore, each of the plurality of metal sheets 10 can have a textured surface to further facilitate the trapping and elimination of the ionized particulates. The plurality of metal sheets 10 can be machined so that the metallic surfaces 11 have roughness, knurls, or patterns. For example, the plurality of metal sheets 10 can have wave patterns formed by shaping several parallel grooves along each metal sheet. The grooves can have different shapes and sizes including, but not limited to, round grooves, triangular grooves, rectangular grooves, or a combination thereof. Further, each of the grooves forming the wave patterns on the plurality of metal sheets 10 can have the same depth. However, each of the grooves may have a different depth. The depth of each of the grooves may be in the range of 0.01 millimeters (mm) to 5 mm, depending on the thickness of the metal sheets as well as the size of the particulates that need to be trapped. In other embodiments, different shapes and patterns can be utilized for the plurality of metal sheets 10.

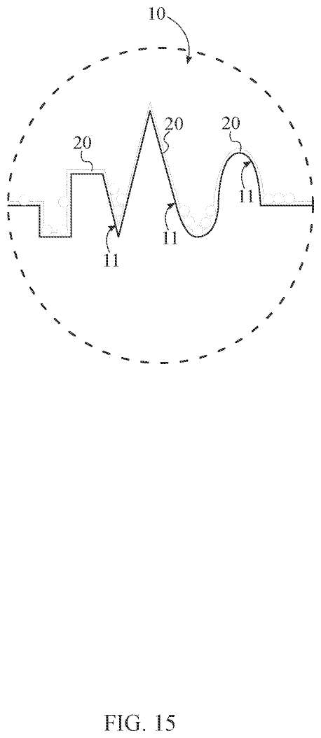

As previously discussed, the metallic air path 8 is electrically connected to the anode 16 of the power source 15 to positively charge the plurality of metal sheets 10. As can be seen in FIG. 15, the positively charged metallic surfaces 11 of the plurality of metal sheets 10 facilitate the attraction and trapping of the ionized particulates from the airflow through the purifier housing 1. To further improve the conductivity of the plurality of metal sheets 10, the present invention may further comprise a quantity of conductive lubricant 20. The quantity of conductive lubricant 20 facilitates the attraction and trapping of the ionized particulates from the airflow through the purifier housing 1. So, the metallic surfaces 11 of the metallic air path 8 are covered by the quantity of conductive lubricant 20. In other embodiments, different lubrication products can be utilized for the metallic air path 8.

In some embodiments, the present invention can allow for different accessories to be inserted into the purifier housing 1 to improve the sanitation of the airflow. As can be seen in FIG. 7 through 12, the present invention may further comprise an air filter 21 that further helps remove pathogens and/or particulates that were not removed from the airflow by the air ionizer 12 or the metallic air path 8. The air filter 21 can be an activated carbon foam pad that removes the ozone produced by the air ionizer 12 as a byproduct. In addition, the purifier housing 1 may comprise at least one insert slot 36 designed to retain the air filter 21. So, the at least one insert slot 36 is positioned adjacent to the at least one air outlet 7 to position the air filter 21 against the at least one air outlet 7. Further, the at least one insert slot 36 is integrated within the purifier housing 1 to secure the at least one insert slot 36 inside the purifier housing 1. Then, the air filter 21 being mounted within the at least one insert slot 36 to secure the air filter 21 inside the at least one insert slot 36. In another embodiment, the present invention may further comprise an air freshener 22 that diffuses aroma on the purified airflow. For example, the air freshener 22 can be an aromatic foam pad that diffuses an aroma for aromatic treatment. The air freshener 22 can be mounted within the same at least one insert slot 36 as the air filter 21. However, a separate insert slot 36 can be provided to hold the air filter 21 and the air freshener 22 separately and adjacent to the at least one air outlet 7.

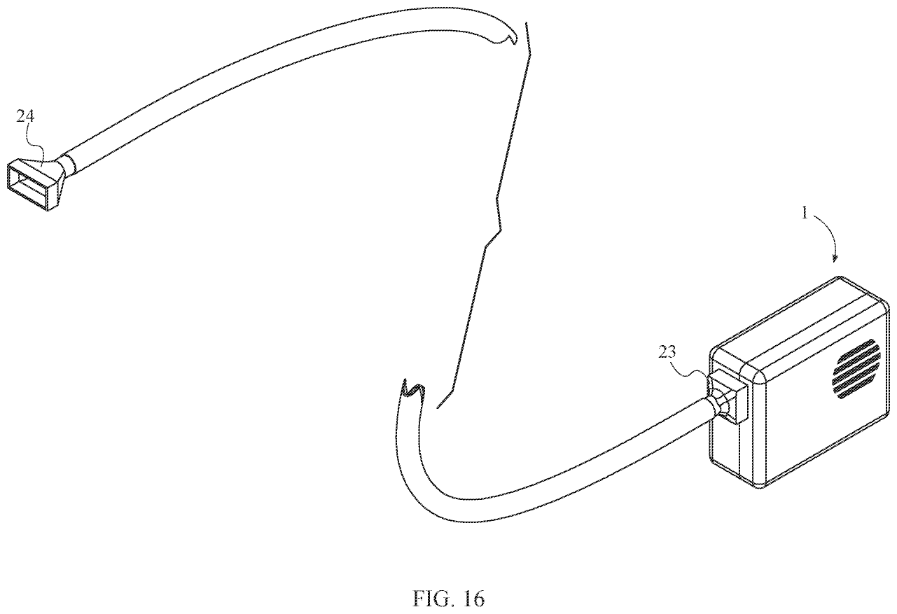

The present invention is preferably designed to be carried by the user on a piece of clothing. The purifier housing 1 can be provided with a fastening attachment to help secure the purifier housing 1 to a piece of clothing. For example, the purifier housing 1 can include a clip that secures the purifier housing 1 to a shirt, belt, strap, etc. Further, the present invention can provide several accessories that facilitate the guidance of the purified airflow coming out of the purifier housing 1 towards the face of the user. As can be seen in FIG. 16, in one embodiment, the present invention may further comprise an air nozzle 24 that guides the outflow of the generated purified air towards the face of the user. The present invention may further comprise a tubing adapter 23 that allows the connection of the air nozzle 24 to the at least one air outlet 7. The air nozzle 24 is positioned externally and offset to the purifier housing 1 so that the air nozzle 24 can be positioned adjacent to the user's face. In addition, the tubing adapter 23 is externally attached to the at least one air outlet 7 to secure the tubing adapter 23 to the at least one air outlet 7. In addition, the air nozzle 24 is in fluid communication with the tubing adapter 23 to guide the purified airflow coming out of the purifier housing 1 towards the user's face via the air nozzle 24. For example, the air nozzle 24 can be connected to at least one air outlet 7 using appropriate tubing. The air nozzle 24 can be designed to guide the outflow of the purified airflow in such a way that the purified airflow surrounds the user's face.

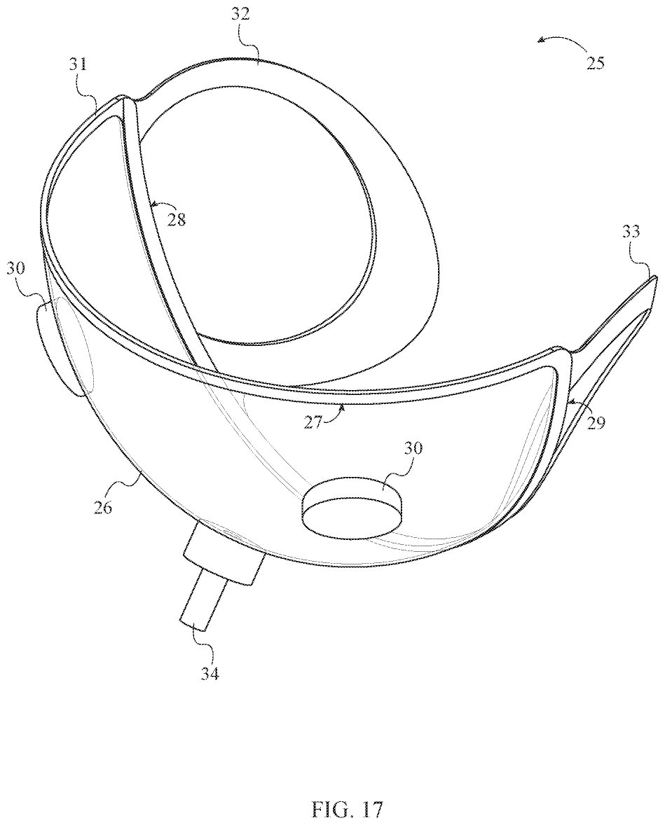

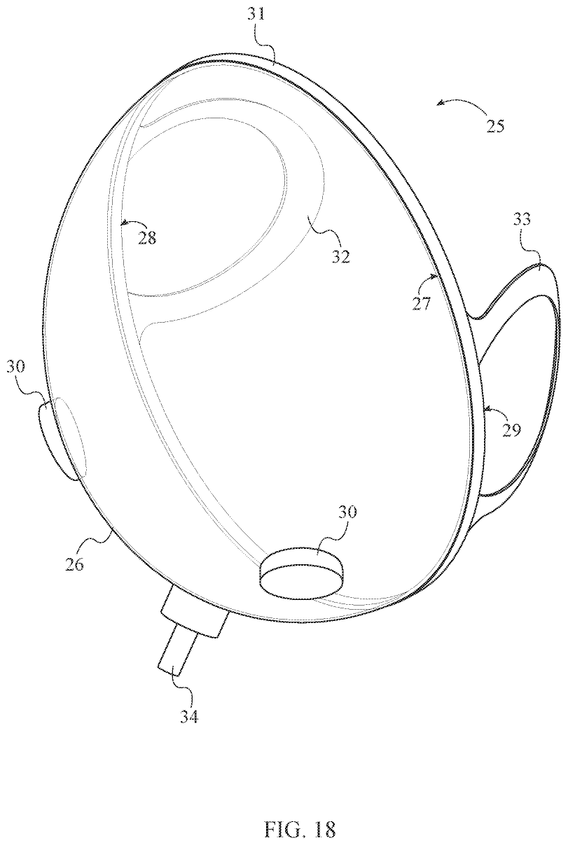

As can be seen in FIGS. 1, 2, 17, and 18, in another embodiment, the present invention may comprise a face mask 25 that guides the purified airflow directly onto the user's face. The face mask 25 further prevents the user from breathing surrounding air and, if the user is sick, from contaminating the surrounding air with pathogens. To do so, the face mask 25 is positioned externally and offset to the purifier housing 1 so that the face mask 25 can be worn on the user's face. Similar to the air nozzle 24, the face mask 25 is in fluid communication with the tubing adapter 23 to guide the purified airflow coming out of the purifier housing 1 towards the user's face via the face mask 25. For example, the face mask 25 can be connected to at least one air outlet 7 using appropriate tubing. In other embodiments, different accessories can be utilized to enable the user to breathe purified air generated by the present invention.

As can be seen in FIGS. 1, 2, 17, and 18, the face mask 25 can include different designs to provide different protection. For example, the face mask 25 can be a partial face mask 25 that covers the user's nose and mouth or a full-face mask 25 that covers the entire face. In general, the face mask 25 may comprise a transparent body 26, a mask inlet 34, at least one air exhaust 30, and an edge trim 31. The transparent body 26 is shaped to contour to the user's face. The mask inlet 34 corresponds to the opening that allows the purified airflow from the purifier housing 1 to flow into the transparent body 26. The at least one air exhaust 30 corresponds to the mechanism that allows the filtration of the user's exhaled air being exhausted into the surroundings. The at least one air exhaust 30 can be equipped with a filter pad, such as a N95 filter, that can be periodically replaced for maintenance. The edge trim 31 provides comfort to the user while also sealing the space formed inside the transparent. The edge trim 31 can be made of soft-silicone rubber or similar rubber materials. Further, the transparent body 26 comprises a perimeter edge 27 corresponding to the outer edge of the transparent body 26. So, the at least one air exhaust 30 and the mask inlet 34 are laterally integrated into the transparent body 26 to secure the at least one air exhaust 30 to the transparent body 26. In addition, the at least one air exhaust 30 and the mask inlet 34 are positioned offset to each other along the transparent body 26. Further, the edge trim 31 is connected along the perimeter edge 27 to secure the edge trim 31 to the perimeter edge 27. Furthermore, the mask inlet 34 is in fluid communication with the tubing adapter 23. In other embodiments, the face mask 25 can be modified to accommodate other features.

As can be seen in FIGS. 1, 2, 17, and 18, furthermore, to facilitate the attachment of the face mask 25 to the user's face, the face mask 25 may further comprise a first ear fastener 32 and a second ear fastener 33. The first ear fastener 32 and the second ear fastener 33 are fastening devices that secure the transparent body 26 to the user's faces. For example, the first ear fastener 32 and the second ear fastener 33 can be ear loops that can be comfortably looped around the user's ears to position the transparent body 26 against the user's face. In addition, the transparent body 26 may comprise a first body end 28 and a second body end 29 corresponding to two opposite ends of the transparent body 26 that match the position of the user's ears. To do so, the first ear fastener 32 is terminally connected to the first body end 28 to secure the first ear fastener 32 to the transparent body 26. Similarly, the second ear fastener 33 is terminally connected to the second body end 29 to also secure the second ear fastener 33 to the transparent body 26. In other embodiments, the face mask 25 can include different features that increase the comfort and safety of the user.

Although the invention has been explained in relation to its preferred embodiment, it is to be understood that many other possible modifications and variations can be made without departing from the spirit and scope of the invention.

What is claimed is:

1. A personal air purifier comprising:
a purifier housing;
at least one air inlet;
at least one air outlet;
a metallic air path;
an air ionizer;
a power source;
an air fan;
a controller;
the at least one air inlet and the at least one air outlet being externally integrated onto the purifier housing;
the at least one air inlet being positioned opposite to the at least one air outlet across the purifier housing;
the metallic air path, the air ionizer, the power source, the air fan, and the controller being mounted within the purifier housing;
the at least one air inlet being in fluid communication with the at least one air outlet through the air fan, through the air ionizer, and through the metallic air path;
the controller being electronically connected to the air ionizer and the air fan;
the power source being electrically connected to the controller, the air ionizer, and the air fan; and
the metallic air path being electrically connected to an anode of the power source.

2. The personal air purifier as claimed in claim 1 comprising:
an air vent; and
the air vent being integrated into the at least one air inlet.

3. The personal air purifier as claimed in claim 1 comprising:
the purifier housing comprising at least one lateral panel and at least one perimeter panel;
the at least one air inlet being integrated into the at least one lateral panel; and
the at least one air outlet being integrated into the at least one perimeter panel.

4. The personal air purifier as claimed in claim 1 comprising:
the purifier housing comprising a first lateral panel and a second lateral panel;
the first lateral panel and the second lateral panel being positioned opposite to each other across the purifier housing;
the at least one air inlet being integrated into the first lateral panel; and
the at least one air outlet being integrated into the second lateral panel.

5. The personal air purifier as claimed in claim 1 comprising:
the air fan comprising a fan housing;
the fan housing being in fluid communication with the at least one air inlet;
the metallic air path being in fluid communication with the at least one air outlet; and
the fan housing being in fluid communication with the metallic air path through the air ionizer.

6. The personal air purifier as claimed in claim 1 comprising:
the air fan comprising a fan housing;
the air ionizer being in fluid communication with the at least one air inlet;
the fan housing being in fluid communication with the at least one air outlet; and
the air ionizer being in fluid communication with the fan housing through the metallic air path.

7. The personal air purifier as claimed in claim 1 comprising:
the air ionizer comprising an ionizer chamber and at least one electrode;
the at least one electrode being mounted within the ionizer chamber; and
the ionizer chamber being in fluid communication with the metallic air path.

8. The personal air purifier as claimed in claim 1 comprising:
the metallic air path comprising a tubular housing and a plurality of metal sheets;
the plurality of metal sheets being mounted within the tubular housing;
each of the plurality of metal sheets being positioned parallel to each other;
each of the plurality of metal sheets being positioned offset to each other; and
the tubular housing being in fluid communication with the air ionizer.

9. The personal air purifier as claimed in claim 8, wherein each of the plurality of metal sheets is made of an oligodynamic metal.

10. The personal air purifier as claimed in claim 8, wherein the tubular housing is made of a plastic material.

11. The personal air purifier as claimed in claim 8, wherein each of the plurality of metal sheets has a textured surface.

12. The personal air purifier as claimed in claim 1 comprising:
a quantity of conductive lubricant; and
metallic surfaces of the metallic air path being covered by the quantity of conductive lubricant.

13. The personal air purifier as claimed in claim 1 comprising:
an air filter;
the purifier housing comprising at least one insert slot;
the at least one insert slot being positioned adjacent to the at least one air outlet;
the at least one insert slot being integrated within the purifier housing; and
the air filter being mounted within the at least one insert slot.

14. The personal air purifier as claimed in claim 13, wherein the air filter is an activated carbon foam pad.

15. The personal air purifier as claimed in claim 1 comprising:
an air freshener;
the purifier housing comprising at least one insert slot;
the at least one insert slot being positioned adjacent to the at least one air outlet;
the at least one insert slot being integrated within the purifier housing; and
the air freshener being mounted within the at least one insert slot.

16. The personal air purifier as claimed in claim 15, wherein the air freshener is an aromatic foam pad.

17. The personal air purifier as claimed in claim 1 comprising:
an air nozzle;
a tubing adapter;
the air nozzle being positioned externally and offset to the purifier housing;
the tubing adapter being externally attached to the at least one air outlet; and
the air nozzle being in fluid communication with the tubing adapter.

18. The personal air purifier as claimed in claim 1 comprising:
a face mask;
a tubing adapter;
the face mask being positioned externally and offset to the purifier housing;
the tubing adapter being externally attached to the at least one air outlet; and
the face mask being in fluid communication with the tubing adapter.

19. The personal air purifier as claimed in claim 18 comprising:
the face mask comprising a transparent body, a mask inlet, at least one air exhaust, and an edge trim;
the transparent body comprising a perimeter edge;
the at least one air exhaust and the mask inlet being laterally integrated into the transparent body;
the at least one air exhaust and the mask inlet being positioned offset to each other along the transparent body;
the edge trim being connected along the perimeter edge; and
the mask inlet being in fluid communication with the tubing adapter.

20. The personal air purifier as claimed in claim 18 comprising:
the face mask comprising a transparent body, a first ear fastener, and a second ear fastener;
the transparent body comprising a first body end and a second body end;
the first ear fastener being terminally connected to the first body end; and
the second ear fastener being terminally connected to the second body end.
